(19) United States
(12) Patent Application Publication
(10) Pub. No.: US 2024/0047074 A1
(43) Pub. Date: Feb. 8, 2024

(54) INFORMATION PROCESSING DEVICE, RECORDING MEDIUM, AND METHOD FOR PROCESSING INFORMATION

(71) Applicants: THE UNIVERSITY OF TOKYO, Tokyo (JP); SIMPLEX QUANTUM INC., Tokyo (JP)

(72) Inventors: Katsuhito FUJIU, Tokyo (JP); Yu SHIMIZU, Tokyo (JP); Issei KOMURO, Tokyo (JP); Eriko HASUMI, Tokyo (JP); Ying CHEN, Tokyo (JP); Ryu SAITO, Tokyo (JP); Minoru SHIRATSUCHI, Tokyo (JP)

(21) Appl. No.: 18/275,168

(22) PCT Filed: Oct. 11, 2022
(86) PCT No.: PCT/JP2022/037794

§ 371 Date: Jul. 31, 2023

(30) Foreign Application Priority Data
Oct. 28, 2021 (JP) 2021-176730

(51) Int. Cl. G16H 50/30; A61B 5/00

(52) U.S. Cl. CPC G16H 50/30; A61B 5/4842; A61B 5/7267; A61B 5/742

(57) ABSTRACT

An information processing device is provided. This information processing device has a read-out unit, a calculation unit, and an output unit. The read-out unit reads a first electrocardiogram waveform of a first subject. The calculation unit calculates an index that depends on the expected value of heart failure severity on the basis of the first electrocardiogram waveform and reference information. The reference information shows the correlation between the index and a second electrocardiogram waveform of a second subject that is different from the first subject. The output unit outputs the index.

### CROSS REFERENCE TO RELATED APPLICATIONS

This application is a U.S. National Phase Application under 35 U.S.C. 371 of International Application No. PCT/JP2022/037794, filed on Oct. 11, 2022, which claims priority to Japanese Patent Application No. 2021-176730, filed on Oct. 28, 2021. The entire disclosures of the above applications are expressly incorporated by reference herein.

### BACKGROUND

The present invention relates to an information processing apparatus, a storage medium, and an information processing method.

### RELATED ART

A heart failure detection method is disclosed in which electrocardiogram, heart rate, heart rate variability, heart rate interval, and respiratory frequency are obtained from a specimen, and information thereof is analyzed to determine whether the specimen has heart failure (see JP 2020-039472 A).

However, with the technique disclosed in JP 2020-039472 A, it is difficult to grasp a worsening status of heart failure due to determination based on two choices of whether it is heart failure.

Given the above circumstances, the present invention provides an information processing apparatus capable of grasping a worsening status of heart failure.

### SUMMARY

According to an aspect of the present invention, an information processing apparatus is provided. The information processing apparatus comprises a reading unit, a calculation unit, and an output unit. The reading unit is configured to read a first electrocardiogram of a first specimen. The calculation unit is configured to calculate an index corresponding to an expected value of heart failure severity based on the first electrocardiogram and reference information. The reference information is information indicating a relationship between a second electrocardiogram of a second specimen, that is different from the first specimen, and the index. The output unit is configured to output the index.

According to the above disclosure, it is possible to grasp a worsening status of heart failure.

### BRIEF DESCRIPTION OF THE DRAWINGS

FIG. 1 is a configuration diagram showing an information processing system 100.

FIG. 2 is a block diagram showing a hardware configuration of an information processing apparatus 200.

FIG. 3 is a block diagram showing a hardware configuration of a user terminal 300.

FIG. 4 is a block diagram showing function realized by the information processing apparatus 200 (controller 210).

FIG. 5 is an activity diagram showing a flow of information processing executed by the information processing apparatus 200.

FIG. 6 is a diagram showing content displayed on a display device of the user terminal 300.

FIG. 7 is a diagram showing a long-term trend of NYHA classification and index for a heart failure patient.

FIG. 8 is a diagram showing distribution of BNP value and corresponding index value.

FIG. 9 is a graph obtained by regression analysis of BNP value and corresponding index value.

FIG. 10 is a diagram showing electrocardiogram of each BNP class.

FIG. 11 is a diagram showing a long-term trend of BNP value and index of a heart failure patient.

FIG. 12 is a diagram showing a long-term trend of BNP value and index of a heart failure patient.

FIG. 13 is a diagram showing a long-term trend of BNP value and index of a heart failure patient.

FIG. 14 is a diagram showing a long-term trend of BNP value and index of a heart failure patient.

### DETAILED DESCRIPTION

Hereinafter, embodiments of the present invention will be described with reference to the drawings. Various features described in the embodiment below can be combined with each other.

A program for realizing a software in the present embodiment may be provided as a non-transitory computer readable medium that can be read by a computer, or may be provided for download from an external server, or may be provided so that the program can be activated on an external computer to realize its functions on a client terminal (so-called cloud computing).

In the present embodiment, the term “unit” may include, for instance, a combination of hardware resources implemented by circuits in a broad sense and information processing of software that can be concretely realized by these hardware resources. Further, although various information is performed in the present embodiment, this information can be represented, for example, by physical signal values representing voltage and current, by high and low signal values as a bit set of binary numbers composed of 0 or 1, or by quantum superposition (so-called quantum bits). In this way, communication/calculation can be performed on a circuit in a broad sense.

Further, the circuit in a broad sense is a circuit realized by combining at least an appropriate number of a circuit, a circuitry, a processor, a memory, and the like. In other words, it is a circuit includes Application Specific Integrated Circuit (ASIC), Programmable Logic Device (e.g., Simple Programmable Logic Device (SPLD), Complex Programmable Logic Device (CPLD), and Field Programmable Gate Array (FPGA)), and the like.

1. Hardware Configuration

In Section 1, a hardware configuration according to the present embodiment will be described.

1.1 Information Processing System 100

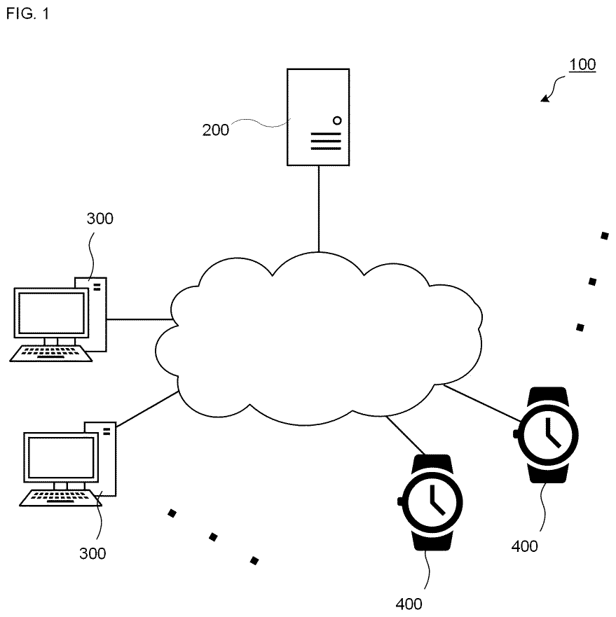

FIG. 1 is a configuration diagram representing an information processing system 100. The information processing system 100 comprises an information processing apparatus 200, a user terminal 300, and an electrocardiograph 400, which are connected through a network. These components will be described further. The system exemplified by the information processing system 100 is one that configured of one or more apparatuses or components. Therefore, for instance, even a single information processing apparatus 200 can be a system exemplified by the information processing system 100.

1.2 Information Processing Apparatus 200

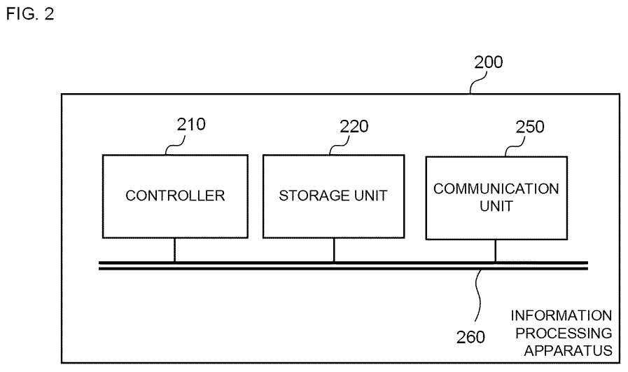

FIG. 2 is a block diagram showing a hardware configuration of the information processing apparatus 200. The information processing apparatus 200 comprises a controller 210, a storage unit 220, and a communication unit 250, and these components are electrically connected inside the information processing apparatus 200 via a communication bus 260. Each component will be described further.

The controller 210 performs processing and control of overall operation related to the information processing apparatus 200. The controller 210 is, for instance, an unshown CPU (Central Processing Unit). The controller 210 is configured to read a predetermined program stored in the storage unit 220 to realize various functions with respect to the information processing apparatus 200. In other words, information processing by software stored in the storage unit 220 can be executed as each function execution unit included in the controller 210 by specifically realized through the controller 210, that is an example of hardware. These will be described in further detail in Section 2. The controller 210 is not limited to being a single controller but may be implemented with a plurality of controllers 210 for each function. Moreover, a combination thereof may be applied.

The storage unit 220 is configured to store various information necessary for information processing of the information processing apparatus 200. This can be implemented, for example, as a storage device such as an SSD (Solid State Drive) that stores various programs, etc. related to the information processing apparatus 200 executed by the controller 210, or as a memory such as an RAM (Random Access Memory) that stores temporarily necessary information (arguments, sequences, etc.) for calculation of program. Further, a combination thereof may be applied.

Although wired communication method such as USB, IEEE1394, Thunderbolt (registered trademark), wired LAN network communication, etc. are preferred, the communication unit 250 may include wireless LAN network communication, mobile communication such as 5G/LTE/3G, Bluetooth (registered trademark) communication, etc. as necessary. That is, it is further preferable to implement as a set of these communication means. In other words, the information processing apparatus 200 communicates various information with the user terminal 300 and the electrocardiograph 400 via the network through the communication unit 250.

1.3 User Terminal 300

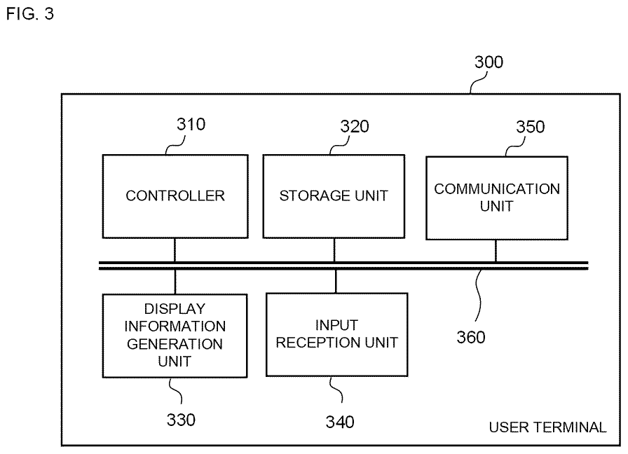

FIG. 3 is a block diagram showing a hardware configuration of the user terminal 300. The user terminal 300 comprises a controller 310, a storage unit 320, a display information generation unit 330, an input reception unit 340, and a communication unit 350, and these components are electrically connected inside the user terminal 300 via a communication bus 360. Descriptions of the controller 310, the storage unit 320, and the communication unit 350 are omitted since they are substantially the same as those of the controller 210, the storage unit 220, and the communication unit 250 in the information processing apparatus 200. The user terminal 300 may be, for instance, a desktop PC, a notebook PC, a smartphone, a tablet terminal, etc.

The display information generation unit 330 is configured to display text, image (including still image and motion image), and generate information to be displayed on a display device such as CRT display, liquid crystal display, organic EL display, plasma display, etc.

The input reception unit 340 is configured to input various information to the user terminal 300 and receive signal input from a mouse, a keyboard, a pointing device, etc. Operation input conducted by a user is transferred as command signal to the controller 310 via the communication bus 360. The controller 310 can then execute predetermined control or calculation as necessary.

1.4 Electrocardiograph 400

The electrocardiograph 400 is configured to acquire data with respect to temporal fluctuation of action potential of cardiac muscle cell accompanying heartbeat as electrocardiogram. The electrocardiograph 400 may be, for instance, a 12-lead electrocardiograph, a wearable terminal with electrocardiograph function, etc. Depending on application, a vector electrocardiograph, a long-time recording electrocardiograph, a body surface potential monitor, an automatic electrocardiograph, etc. may be selected as appropriate.

The electrocardiograph 400 is connected to the communication unit 250 in the information processing apparatus 200 via a network, and is configured to transfer the acquired electrocardiogram to the information processing apparatus 200. The electrocardiograph 400 may not include a communication unit. In this case, the acquired electrocardiogram may be stored in a storage medium such as a memory card, transferred from the storage medium to the user terminal 300, and then transferred from the user terminal 300 to the information processing apparatus 200.

2. Functional Configuration

In Section 2, a functional configuration according to the present embodiment will be described. As mentioned above, information processing by software stored in the storage unit 220 can be executed as each function execution unit included in the controller 210 by specifically realized through the controller 210, that is an example of hardware.

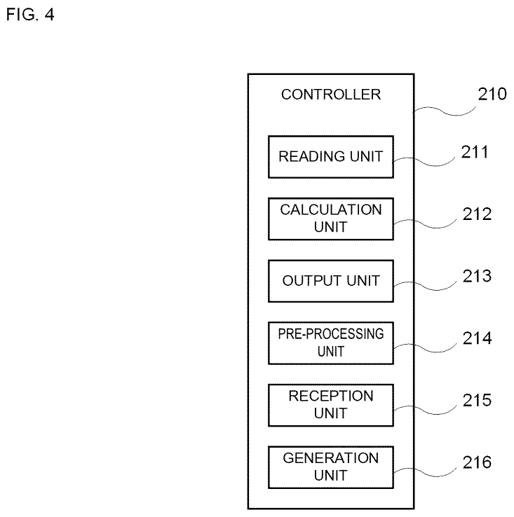

FIG. 4 is a block diagram showing function realized by the information processing apparatus 200 (controller 210). Specifically, information processing apparatus 200 (controller 210) comprises a reading unit 211, a calculation unit 212, an output unit 213, a pre-processing unit 214, a reception unit 215, and a generation unit 216.

The reading unit 211 is configured to read various information. For example, the reading unit 211 is configured to read a first electrocardiogram of a first specimen, that is an arbitrary specimen.

The calculation unit 212 is configured to calculate various information. For instance, the calculation unit 212 is configured to calculate an index corresponding to an expected value of heart failure severity based on the read first electrocardiogram and reference information. Here, the reference information is information indicating a relationship between a second electrocardiogram of a second specimen, that is different from the first specimen, and the index.

The output unit 213 is configured to output various information. For example, the output unit 213 is configured to output the calculated index.

The pre-processing unit 214 is configured to pre-process various information. For example, the pre-processing unit 214 is configured to pre-process the read first electrocardiogram before it is calculated and processed by the calculation unit 212. Here, the pre-processing is at least one of trend removal, motion artifact removal, noise removal, waveform data cutout for each heartbeat, and normalization.

The reception unit 215 is configured to receive various information. For example, the reception unit 215 is configured to receive the first electrocardiogram acquired by the electrocardiograph 400.

The generation unit 216 is configured to generate various information. For instance, the generation unit 216 is configured to generate visual information with respect to the index that is visible at the user terminal 300.

3. Information Processing Method

In Section 3, an information processing method of the information processing apparatus 200 will be described. The information processing method is an information processing method executed by a computer. The information processing method comprises a reading step, a calculating step, and an outputting step. In the reading step, a first electrocardiogram of a first specimen, that is an arbitrary specimen, is read. In the calculating step, an index is calculated according to an expected value of heart failure severity based on the first electrocardiogram and reference information. The reference information is information that indicates a relationship between a second electrocardiogram of a second specimen, that is different from the first specimen, and the index. In the outputting step, the index is output.

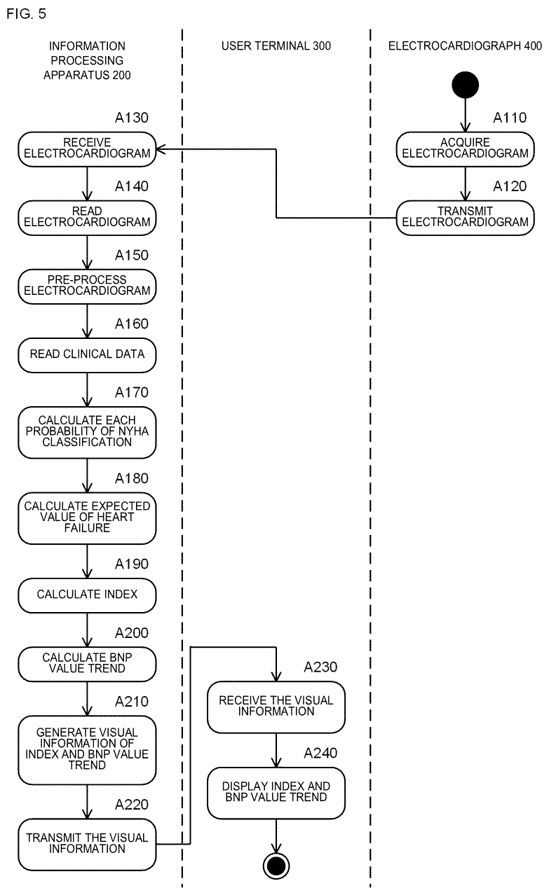

FIG. 5 is an activity diagram showing a flow of information processing executed by the information processing apparatus 200. Following description shall follow each activity in the activity diagram.

The electrocardiograph 400 acquires the first electrocardiogram of the first specimen being an arbitrary specimen (Activity A110).

The first electrocardiogram may be configured of 1 to 50 beats. Specifically, the first electrocardiogram may be configured of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 43, 43, 44, 45, 46, 47, 48, 49, 50 beats, and may be within a range between any two of the numerical values illustrated here. Preferably, the first electrocardiogram is configured of 1 to 2 beats.

The first electrocardiogram may be configured of a waveform in 5 to 300 seconds. Specifically, for instance, the first electrocardiogram may be configured of a waveform in 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300 seconds, and may be within a range between any two of the numerical values illustrated here. Preferably, the first electrocardiogram is configured of a waveform in 5 to 10 seconds.

The first electrocardiogram may be acquired from an electrode corresponding to any one of 12 leads. Preferably, the one lead is lead I acquired from right and left hands of the first specimen.

The electrocardiograph 400 then transmits the acquired first electrocardiogram to the information processing apparatus 200 via a network (Activity A120).

The reception unit 215 then receives the first electrocardiogram transmitted from the electrocardiograph 400 (Activity A130). That is, the communication unit 250 in the information processing apparatus 200 receives the first electrocardiogram, and the controller 210 has the storage unit 220 stores the received first electrocardiogram.

The reading unit 211 then reads the first electrocardiogram of the first specimen (Activity A140). That is, the controller 210 accesses the storage unit 220 and reads the first electrocardiogram stored in the storage unit 220 by executing reading processing.

The pre-processing unit 214 then pre-processes the read first electrocardiogram (Activity A150). That is, the controller 210 reads predetermined parameter stored in the storage unit 220 and applies the parameter to raw data of the first electrocardiogram, thereby processing the first electrocardiogram. Here, the pre-processing may be, for instance, trend removal, motion artifact removal, noise removal, waveform data cutout for each heartbeat, normalization, etc., and a combination thereof may be executed as appropriate. Preferably, the pre-processing is executed in following order: trend removal, motion artifact removal, noise removal, waveform data cutout for each heartbeat, and normalization.

The reading unit 211 then reads clinical data of the first specimen (Activity A160). That is, the controller 210 accesses the storage unit 220 and reads the clinical data of the first specimen stored in the storage unit 220 by executing reading processing. Here, the clinical data may be data including at least one of age, gender, BMI (Body Mass Index), PWTT (Pulse Wave Transit Time), blood pressure, heart rate, SDNN (Standard Deviation of the Normal-to-Normal Interval), CVRR (Coefficient of Variation of R-R Interval), atrial fibrillation, and HRV (Heart Rate Variability).

The calculation unit 212 then calculates an index corresponding to an expected value of heart failure severity based on the first electrocardiogram of the first specimen, the clinical data of the first specimen, and the reference information (Activity A170 to Activity A190). That is, controller 210 reads the reference information stored in the storage unit 220 and calculates the index corresponding to the expected value of heart failure severity by executing the calculation process based on the pre-processed first electrocardiogram, the read clinical data of the first specimen, and the reference information.

Here, the reference information is information indicating a relationship between the second electrocardiogram of the second specimen, that is different from the first specimen, clinical data of the second specimen, and the index according to the expected value of heart failure severity. Preferably, the reference information is a learned model in which relation between the second electrocardiogram, the clinical data of the second specimen and the index is previously learned as teaching data. The second electrocardiogram is obtained in advance and indicates an electrocardiogram for which heart failure severity has been diagnosed by a physician. In the present embodiment, the reference information is described as a learned model.

Processing from Activity A170 to Activity A190 will be described in turn. The calculation unit 212 calculates each probability of NYHA classification (Activity A170). That is, the controller 210 reads the pre-processed first electrocardiogram, the clinical data of the first specimen and the reference information from the storage unit 220, and calculates each probability of NYHA classification by executing calculation processing.

The NYHA classification is a classification of heart failure created by NYHA (New York Heart Association) and indicates four-stage classifications of heart failure severity based on degree of subjective symptom caused by physical activity.

The calculation unit 212 then calculates an expected value of heart failure based on each probability of NYHA classification (Activity A180). That is, the controller 210 reads following Equation (1) stored in the storage unit 220 and calculates the expected value of heart failure by substituting the value calculated in Activity A170 into Equation (1).

$$[ Equation 1 ]$$

$$E [ X ] = {\sum }_{i = 1}^{5} {x}_{i} P ( X = {x}_{i} ) , x ∈ \{ 0 , 1 , 2 , 3 , 4 \} \qquad ( 1 )$$

Here, E indicates expected value of heart failure, and x indicates NYHA classification.

The calculation unit 212 then calculates an index based on the expected value of heart failure (Activity A190). That is, the controller 210 reads following Equation (2) stored in the storage unit 220 and calculates the index by substituting the value calculated in Activity A180 into Equation (2). In the present embodiment, the index ranges from 0 to 100.

$$[ Equation 2 ]$$

$$Index = \frac{E [ X ] - {x}_{min}}{( {x}_{max} - {x}_{min} )} \times 100 , 0 ≦ Index ≦ 100 \qquad ( 2 )$$

The calculation unit 212 then calculates a trend of BNP value from the index (Activity A190). That is, the controller 210 reads a predetermined equation stored in the storage unit 220 and calculates the trend of BNP value by substituting the value calculated in Activity A190 into the equation.

Here, BNP indicates cerebral (B-type) natriuretic peptide, and shows action of expanding blood vessel and promoting urine drainage. In other words, BNP has physiological effect of relieving stress on heart, and thus reflects a status of heart failure according to concentration in blood. Therefore, BNP can be used as a biomarker for heart failure.

The generation unit 216 then generates visual information with respect to the index and BNP value that is visible at the user terminal 300 (Activity A210). In other words, the controller 210 reads the index and BNP value from the storage unit 220 and executes generation processing to generate visual information with respect to the index and BNP value.

The output unit 213 then outputs the visual information with respect to the index and BNP value (Activity A220). In other words, the controller 210 processes transmission of the generated visual information to the user terminal 300 via the communication unit 250.

The controller 310 in the user terminal 300 then processes reception of the visual information transmitted from the information processing apparatus 200 via the communication unit 350 (Activity A230).

The controller 310 then displays the visual information with respect to the received index and BNP value on a display device of the user terminal 300 (Activity A240).

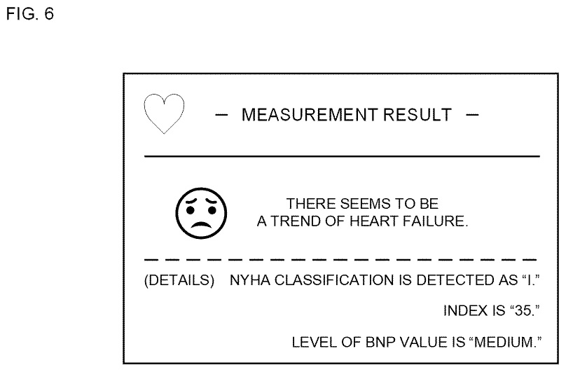

FIG. 6 shows content displayed on the display device of the user terminal 300. In FIG. 6, it is assumed that the probability that the NYHA classification of the first specimen is “degree I” is 60% and the probability that the NYHA classification is “degree II” is 40%. In this case, since the probability of NYHA classification “degree I” is higher than the probability of NYHA classification “degree II,” the NYHA classification of the first specimen is detected as “degree I.” Subsequently, the expected value of heart failure is calculated as “1.4” by substituting the NYHA classification value “I” into Equation (1). The index is then calculated as “35” by substituting the expected value of heart failure “1.4” into Equation (2). The trend of the BNP value is then calculated as “medium” by substituting the index value “35” into a predetermined equation.

Contents shown in FIG. 6 indicate the following. For instance, if the probability of NYHA classification being “degree I” is 100% (pattern 1), the severity of heart failure is NYHA classification “degree I”. On the other hand, if the probability of NYHA classification being “degree I” is 60% and that of NYHA classification being “degree II” is 40% (pattern 2), the severity of heart failure is between NYHA classification “degree I” and “degree II,” but indicated as NYHA classification “degree I.” Here, if the severity of heart failure is indicated by the index according to the present embodiment, the index is “25” in pattern 1, and is “35” in pattern 2. Accordingly, it can be indicated that the heart failure situation in pattern 2 is worse than that in pattern 1.

Therefore, since the index according to the present embodiment shows more detailed information than the NYHA classification, it can capture worsening status of heart failure of a specimen at an early stage, and can assist a physician in diagnosis and lead to early treatment as well. In addition, since the index can be realized in a variety of electrocardiographs 400, it can be useful for prevention of heart failure and home monitoring of heart failure patients after leaving hospital.

4. Usefulness of Index

Section 4 describes the usefulness of the index output in the present embodiment.

4.1 Correlation Between NYHA Classification and Index

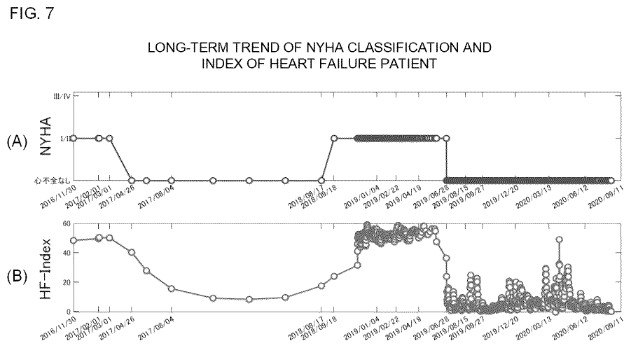

FIG. 7 shows a long-term trend between the NYHA classification and index for a heart failure patient. FIG. 7(A) shows a long-term trend of the NYHA classification and FIG. 7(B) shows a long-term trend of the index.

NYHA classification shows “degree I” or “degree II” from Nov. 30, 2016, to Mar. 1, 2017. Index shows “50” from Nov. 30, 2016, to Mar. 1, 2017. NYHA classification shows “no heart failure” from Apr. 26, 2017, to Aug. 17, 2018. Index shows “40” as of Apr. 26, 2017, and shows “20” or less from Aug. 4, 2017, to Aug. 17, 2018.

NYHA classification shows “degree I” or “degree II” from Sep. 18, 2018, to Jun. 28, 2019. Index shows an upward trend from Sep. 18, 2018, to Jun. 28, 2019, showing “40” to “60”. NYHA classification shows “no heart failure” from Jun. 28, 2019, to Sep. 11, 2020. Index shows an abbreviation of “20” or less from Jun. 28, 2019, to Sep. 11, 2020.

As described above, the index according to the present embodiment is shown to be correlated with the NYHA classification.

4.2 Correlation Between BNP Value Trend and Index

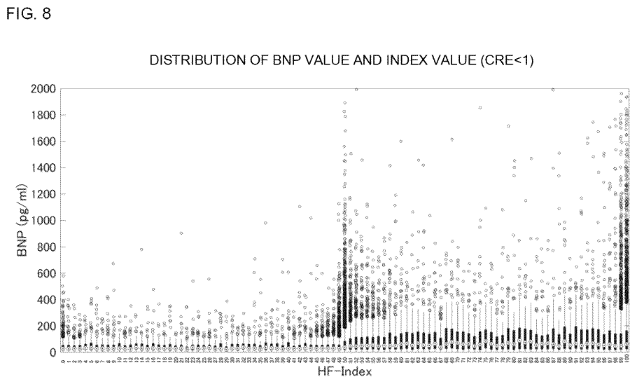

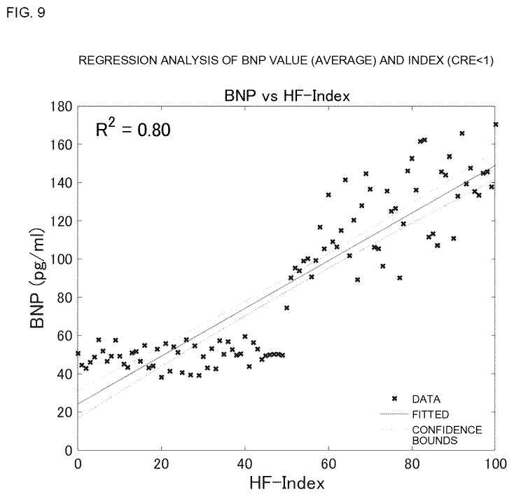

FIG. 8 shows a distribution of BNP value and corresponding index value. FIG. 9 shows a regression analysis of BNP value and corresponding index value. In FIGS. 8 and 9, it can be read that there is a positive correlation between the BNP value and the corresponding index value.

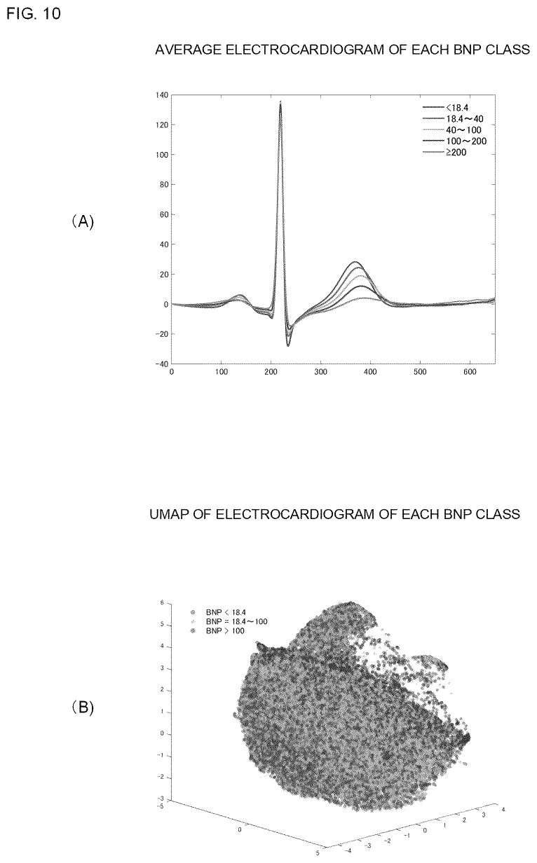

FIG. 10 shows electrocardiogram of BNP for each class. FIG. 10(A) shows average electrocardiogram and FIG. 10(B) shows UMAP of electrocardiogram. In FIG. 10, BNP values are divided into several classes and corresponding electrocardiograms for each class are analyzed. As a result, it can be read that the electrocardiogram in FIG. 10 varied according to the BNP value.

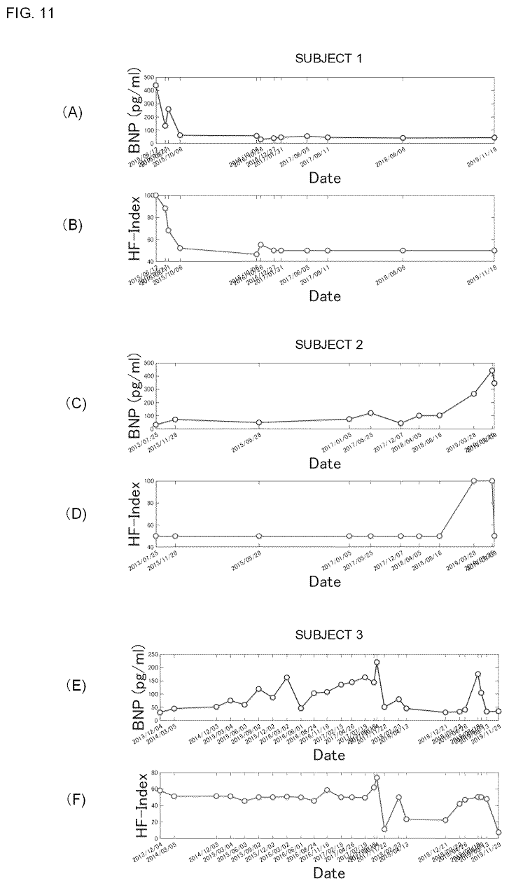

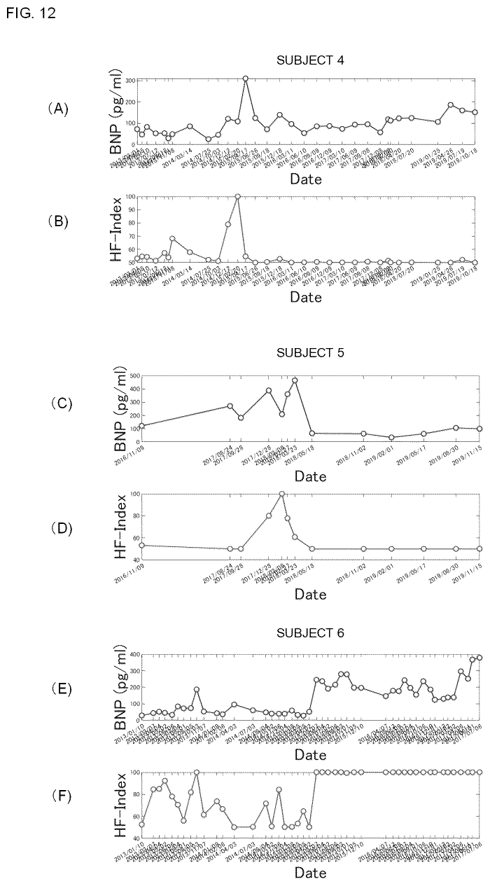

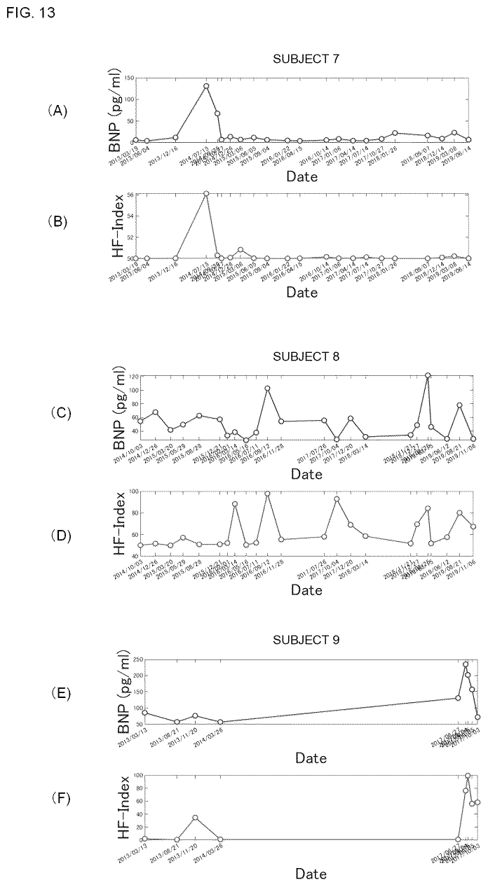

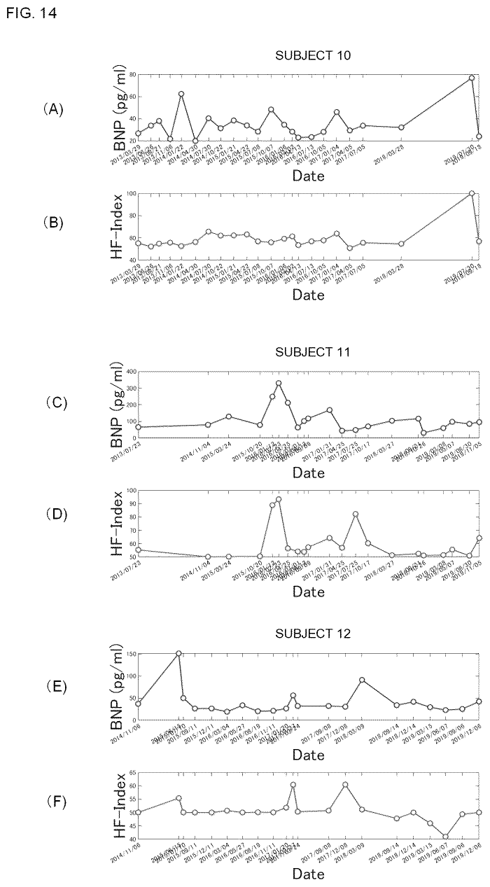

FIGS. 11 to 14 show long-term trend between BNP value and index of heart failure patient. In FIG. 11, a long-term trend between BNP value and index of three heart failure patients (subjects 1 to 3) is divided into (A) to (F). In FIG. 12, a long-term trend between BNP value and index of three heart failure patients (subjects 4 to 6) is divided into (A) to (F). In FIG. 13, a long-term trend between BNP value and index of three heart failure patients (subjects 7 to 9) is divided into (A) to (F). In FIG. 14, a long-term trend between BNP value and index of three heart failure patients (subjects 10 to 12) is divided into (A) to (F).

In FIGS. 11 to 14, blood sampling is performed on 12 heart failure patients to measure BNP value, and the information processing apparatus 200 is applied to calculate index. As a result, it can be read that there is a positive correlation between the BNP value and the corresponding index value in FIGS. 11 to 14.

According to the above, it has been demonstrated that the index output in the present embodiment is correlated with heart failure severity. Therefore, the index can be suitably applied to detect heart failure severity.

An implement aspect of the present embodiment may be a program. The program causes a computer to function as each part of the information processing apparatus 200.

An implement aspect of the present embodiment may be a storage medium. The storage medium stores a program that causes a computer to function as each part of the information processing apparatus 200.

Although the embodiments of the invention have been described above, the present invention is not limited thereto and can be modified as appropriate without departing from the technical concept of the present invention.

As a first variation, output by the output unit 213 is not limited to transmitting visual information related to the trend of the index and the BNP value to the user terminal 300, and may be, for instance, displayed on a display device of the information processing apparatus 200.

As a second variation, the index according to the expected value of heart failure severity is not limited to a range of 0 to 100 and may be, for example, in a range of 0 to 1 or 0 to 10. Depending on range of index, Equation (2) can be appropriately set accordingly.

As a third variation, the reference information is not limited to a learned model, and may be, for example, a database that stores look-up table, etc., a function that represents correspondence of value determined depending on certain variable, a mathematical model that mathematically relates a plurality of pieces of information, or the like.

As a fourth variation, the electrocardiograph 400 may be configured to communicate with the information processing apparatus 200 via the user terminal 300. In this case, the electrocardiograph 400 may be connected to the communication unit 350 in the user terminal 300 via a network, or it may be implemented to connect directly with the user terminal 300.

As a fifth variation, the output unit 213 may be configured to further output other items in addition to index and BNP value trend. For instance, the output unit 213 may further output blood sampling result of the first specimen. The blood sampling result is not limited, and may include, for example, liver test such as AST and ALT, hepatitis test such as HBs antigen and HCV antibody, lipid test such as total cholesterol and triglyceride, sugar metabolism test such as blood sugar and HbA1c, kidney and spleen test such as creatinine and amylase, and blood cell count such as white blood cell count and red blood cell count, etc.

As a sixth variation, the calculation unit 212 may calculate index according to expected value of heart failure severity based on the first electrocardiogram of the first specimen and the reference information, without using the clinical data of the first specimen.

As a seventh variation, the reference information may be information indicating a relationship between the second electrocardiogram of the second specimen and index according to the expected value of heart failure severity, regardless of the clinical data of the second specimen. In this case, the reference information may be a learned model in which a relationship between the second electrocardiogram and the index is previously learned as teaching data.

As an eighth variation, the calculation unit 212 is not limited to calculating the index based on the probability of each NYHA classification, but may also calculate index based on other assessments of heart failure severity (e.g., ACC/AHA classification).

The present invention may be provided in each of the following aspects.

(1) An information processing apparatus, comprising: a reading unit configured to read a first electrocardiogram of a first specimen; a calculation unit configured to calculate an index corresponding to an expected value of heart failure severity based on the first electrocardiogram and reference information, wherein the reference information is information indicating a relationship between a second electrocardiogram of a second specimen, that is different from the first specimen, and the index; and an output unit configured to output the index.

According to such an aspect, since stage information is increased more than conventional NYHA classification, worsening status of heart failure can be grasped before subjective symptoms are present. In addition, processing speed of a computer can be increased due to a simple configuration.

(2) The information processing apparatus according to (1), wherein: the reference information is a learned model in which the relationship between the second electrocardiogram and the index is pre-learned as teaching data.

According to such an aspect, worsening status of heart failure can be grasped with higher accuracy by machine learning.

(3) The information processing apparatus according to (1), wherein: the calculation unit is configured to calculate the index based on the first electrocardiogram, clinical data of the first specimen, and the reference information, and the reference information is information indicating a relationship between the second electrocardiogram, the clinical data of the second specimen, and the index.

According to such an aspect, further use of clinical data makes it possible to grasp worsening status of heart failure with higher accuracy.

(4) The information processing apparatus according to (3), wherein: the reference information is a learned model in which the relationship between the second electrocardiogram, the clinical data of the second specimen, and the index is pre-learned as teaching data.

According to such an aspect, worsening status of heart failure can be grasped with higher accuracy by machine learning.

(5) The information processing apparatus according to (3) or (4), wherein: the clinical data is data including at least one of age, gender, BMI (Body Mass Index), PWTT (Pulse Wave Transit Time), blood pressure, heart rate, SDNN (Standard Deviation of the Normal-to-Normal Interval), CVRR (Coefficient of Variation of R-R Interval), atrial fibrillation, and HRV (Heart Rate Variability).

According to such an aspect, various clinical data can be used to grasp worsening status of heart failure with higher accuracy.

(6) The information processing apparatus according to any one of (1) to (5), comprising: a pre-processing unit configured to pre-process the first electrocardiogram, wherein the pre-process is at least one of trend removal, motion artifact removal, noise removal, waveform data cutout for each heartbeat, and normalization.

According to such an aspect, by removing noise from the first electrocardiogram, worsening status of heart failure can be grasped with higher accuracy.

(7) The information processing apparatus according to any one of (1) to (6), wherein: the first electrocardiogram is configured of 1 to 50 beats.

According to such an aspect, it is possible to grasp worsening state of heart failure in a shorter time.

(8) The information processing apparatus according to any one of (1) to (6), wherein: the first electrocardiogram is configured of a waveform in a range of 5 to 300 seconds.

According to such an aspect, it is possible to grasp worsening state of heart failure in a shorter time.

(9) The information processing apparatus according to any one of (1) to (8), wherein: the first electrocardiogram is acquired from an electrode corresponding to any one of 12 leads.

Such an aspect allows for more types of electrocardiographs to be used, making it easier to achieve home monitoring of heart failure.

(10) The information processing apparatus according to (9), wherein: the one lead is lead I.

According to such an aspect, since the electrocardiogram can be easily acquired, home monitoring of heart failure can be easily realized.

(11) The information processing apparatus according to any one of (1) to (10), comprising: a reception unit configured to receive the first electrocardiogram, and a generation unit configured to generate visual information with respect to the index that is visible at a user terminal.

According to such an aspect, information on worsening status of heart failure can be provided via a network such as the Internet.

(12) The information processing apparatus according to any one of (1) to (11), wherein: the calculation unit is configured to calculate a trend of BNP value from the index.

According to such an aspect, by further calculating information on heart failure, worsening situation of heart failure can be grasped in a more multifaceted manner.

(13) A non-transitory computer readable storage medium, wherein: the storage medium stores a program allowing a computer to function as each part of the information processing apparatus according to any one of (1) to (12).

According to such an aspect, since stage information is increased more than conventional NYHA classification, worsening status of heart failure can be grasped before subjective symptoms are present. In addition, processing speed of a computer can be increased due to a simple configuration.

(14) An information processing method executed by a computer, comprising: a reading step of reading out a first electrocardiogram of a first specimen; a calculating step of calculating an index corresponding to an expected value of heart failure severity based on the first electrocardiogram and reference information, wherein the reference information is information indicating a relationship between a second electrocardiogram of a second specimen, that is different from the first specimen, and the index; and an outputting step of outputting the index.

According to such an aspect, since stage information is increased more than conventional NYHA classification, worsening status of heart failure can be grasped before subjective symptoms are present. In addition, processing speed of a computer can be increased due to a simple configuration.

Of course, the present invention is not limited to the above aspects.

What is claimed is:

1. An information processing apparatus, comprising:
a memory configured to store a program; and
a processor configured to execute the program so as to
read a first electrocardiogram of a first specimen;
calculate each probability of heart failure severity based on the first electrocardiogram and reference information,
calculate an expected value of heart failure severity based on the each probability of heart failure severity and a first equation, and
calculate an index corresponding to the expected value of heart failure severity based on the expected value of heart failure severity and a second equation different from the first equation, wherein
the reference information is information indicating a relationship between a second electrocardiogram of a second specimen, that is different from the first specimen, and the index; and
output the index.

2. The information processing apparatus according to claim 1, wherein:
the reference information is a learned model in which the relationship between the second electrocardiogram and the index is pre-learned as training data.

3. The information processing apparatus according to claim 1, wherein:
the processor is configured to execute the program so as to calculate the index based on the first electrocardiogram, clinical data of the first specimen, and the reference information, and
the reference information is information indicating a relationship between the second electrocardiogram, the clinical data of the second specimen, and the index.

4. The information processing apparatus according to claim 3, wherein:
the reference information is a learned model in which the relationship between the second electrocardiogram, the clinical data of the second specimen, and the index is pre-learned as training data.

5. The information processing apparatus according to claim 3, wherein:
the clinical data is data including at least one of age, gender, BMI (Body Mass Index), PWTT (Pulse Wave Transit Time), blood pressure, heart rate, SDNN (Standard Deviation of the Normal-to-Normal Interval), CVRR (Coefficient of Variation of R-R Interval), atrial fibrillation, and HRV (Heart Rate Variability).

6. The information processing apparatus according to claim 1, wherein:
the processor is configured to execute the program so as to pre-process the first electrocardiogram, wherein
the pre-process is at least one of trend removal, motion artifact removal, noise removal, heartbeat waveform segmentation, and normalization.

7. The information processing apparatus according to claim 1, wherein:
the first electrocardiogram is configured of 1 to 50 beats.

8. The information processing apparatus according to claim 1, wherein:
the first electrocardiogram is configured of a waveform in a range of 5 to 300 seconds.

9. The information processing apparatus according to claim 1, wherein:
the first electrocardiogram is acquired from an electrode corresponding to any one of 12 leads.

10. The information processing apparatus according to claim 9, wherein:
the one lead is lead I.

11. The information processing apparatus according to claim 1, wherein:
the processor is configured to execute the program so as to receive the first electrocardiogram, and
generate visual information with respect to the index that is visible at a user terminal.

12. The information processing apparatus according to claim 1, wherein:
the processor is configured to execute the program so as to calculate a trend of BNP value from the index.

13. A non-transitory computer readable storage medium, wherein:
the storage medium stores a program allowing a computer to function as each part of the information processing apparatus according to claim 1.

14. An information processing method executed by a computer, comprising:
a reading step of reading a first electrocardiogram of a first specimen;
a calculating step of
calculating each probability of heart failure severity based on the first electrocardiogram and reference information,
calculating an expected value of heart failure severity based on the each probability of heart failure severity and a first equation, and
calculating an index corresponding to the expected value of heart failure severity based on the expected value of heart failure severity and a second equation different from the first equation, wherein
the reference information is information indicating a relationship between a second electrocardiogram of a second specimen, that is different from the first specimen, and the index; and
an outputting step of outputting the index.
